(19) United States
(12) Patent Application Publication
(10) Pub. No.: US 2011/0301675 A1
(43) Pub. Date: Dec. 8, 2011

(54) NATURAL HOT WATER BOTTLE SUBSTITUTE

(76) Inventor: Hagay Mizrahi, Chatsworth, CA (US)

(21) Appl. No.: 12/795,616

(22) Filed: Jun. 7, 2010

(51) Int. Cl. A61F 7/00; A61L 9/015

(52) U.S. Cl. 607/114; 239/34

(57) ABSTRACT

A natural hot water bottle substitute includes a container formed of flexible, porous material. The container has a front wall, a back wall and a handle located at an upper end of the front and back walls. A plurality of fastenings attaches the front wall to the back wall at a series of point locations while providing a continuous open space around the fastenings. Temperature retaining material is located within the open space. A fitted cover has a front panel, a rear panel and an access covering panel. The rear panel is similar to the front panel and attached to the front panel at the top end, and along side edges of the front panel, leaving an opening along the bottom end covered by the access covering panel. The access covering panel covers the opening along the bottom end and secures the natural hot water bottle substitute within the cover.

### FIELD OF INVENTION

This invention relates to the field of therapeutic, body soothing devices and more specifically, to devices that incorporate heat retaining substances and aromatherapy products that can be heated or cooled and placed upon the body for soothing effects.

### BACKGROUND OF THE INVENTION

Hot water bottles have long been used to soothe aches and pains through the delivery of localized heating to sensitive or painful areas of the body. The use of these devices, however, is less than convenient. The hot water bottle must be carefully filled with hot water and sealed to prevent leakage. Often, the rubber or similar surface of the bottle is too hot to be placed upon the skin directly until the water bottle cools somewhat. This usually is dealt with by wrapping or encasing the hot water bottle in some type of protective cover. This cover is usually removed after the hot water bottle cools to some extent, a further inconvenience. Further, a standard hot water bottle can not be comfortably used to provide cooling effects without risk of damage to the bottle and potential injury to the user. An improved hot water bottle would address these concerns and provide enhanced comfort and therapeutic benefit in use.

A number of substitutes for typical hot water bottles have been developed as shown in the following references.

U.S. Patent Application No. 2009/0048650, published for Junkins, discloses a configurable ice pack and heating pad. The apparatus resembles a “hot water” bottle and comprises a filler portion and an interior space that receives filler material. The apparatus has a contoured shape and is made of a polyester or plastic material that allows the device to flex and fit around various body parts of the individual using the apparatus. The filler material may be inserted into the filler portion through opening and may be a liquid such as water or a solid such as beans or rice. After the chosen filler material is inserted into the apparatus, the apparatus may be heated in a microwave and then the device is placed on a particular portion of the user's body in order to provide a source of heat in order to manage pain or relieve stress. The device may be used for both hot and cold treatments of minor pain.

U.S. Pat. No. 5,890,487, issued to Kimmel is directed to a corn filled heating pad that is made of a fabric material and is designed to hold a quantity of dried Indian corn. The filled device may then be heated in a microwave oven or cooled in a freezer to provide heat or cooling therapy for an extended period of time. The fabric bag acts as a thermal shield in order to protect the user from excessive temperatures produced by the material held within. The device may include a plurality of baffle stitches within the bag. These stitches form a plurality of sections that keep the corn spread evenly throughout the bag. The various embodiments may be formed so that they are convenient to use around a number of portions of the body such as a user's shoulders, neck, waist, back, or a joint area such as a knee, ankle, or elbow.

U.S. Pat. No. 6,699,271, issued to Clayton illustrates therapeutic wraps and may be formed to accommodate various body portions of an individual such as the neck or shoulders. The wrap contains a plurality of narrow channels into which a filler material may be placed. In order to utilize the device for therapeutic purposes, the filled device is placed into a conventional or microwave oven to be heated, or may be placed in a refrigerator or freezer in order to be cooled so that the device may be used for both hot and cold therapy. The materials used within the device may be Basmati rice and herbs such as chamomile, lavender or other relaxing herbs that may also be used for aromatherapy purposes.

U.S. Pat. No. 7,125,417, issued to Mizrahi is directed to a therapeutic upper body wrap and an aromatherapy herb pack. The devices seen in these two references are designed to fit over specific areas of a person's body and both are divided into a plurality of subspaces in order to prevent shifting of the contained materials. It is intended that the devices may be used to either heat or cool various body parts, but if the choice of material includes herbs, it may be used for aromatherapy purposes. The materials used within the devices include corn, rice, wheat, oats, barley, beans, and flax seed, as well as aromatherapy materials such as cinnamon, chamomile, lemongrass, peppermint, rosemary, spearmint, valerian root, white willow, yarrow, lavender ginger, and yellow dark root.

U.S. Pat. No. 5,375,278, issued to VanWinkle et al. is directed to a therapeutic pillow and method. The pillow has a baglike cover that is filled with a natural granular material and is used for treating pain by heating or cooling the device and then applying it to areas of a body for therapeutic purposes. The baglike covering structure may be held within a removable outer sleeve which may be removed in order to wash same or to control heat transfer properties of the device.

It is an objective of the present invention to provide a device that can be conveniently heated or cooled and used to transfer heat or cooling to the body of a user without fear of burning or unduly chilling the skin of the user. It is a further objective to provide such capabilities without the need for liquid water that may leak upon the user. It is a still further objective of the invention to provide aromatherapy benefits to the user. It is yet a further objective to allow the user to renew the aromatherapy ingredients of the device as required. Finally, it is an objective of the present invention to provide a cover that will help the device to retain the heat or cold of the device for longer periods of time.

While some of the objectives of the present invention are disclosed in the prior art, none of the inventions found include all of the requirements identified.

### SUMMARY OF THE INVENTION

The present invention addresses all of the deficiencies of prior art natural hot water bottle substitute inventions and satisfies all of the objectives described above.

(1) A natural hot water bottle substitute providing all of the desired features can be constructed from the following components. A container formed of flexible, porous material is provided. The container has a front wall, a back wall, a first predetermined length, a first predetermined width, a first predetermined thickness and a handle portion located at an upper end of the front and back walls. A plurality of fastenings is provided. The fastenings attach the front wall to the back wall at a series of point locations while providing a continuous open space around the fastenings. Granular, temperature retaining material is provided. The material is located within the continuous open space. Stitching is provided. The stitching fastens a surrounding edge of the front wall to a surrounding edge of the back wall and encloses the temperature retaining material.

(2) In a variant of the invention, the temperature retaining material is selected from the group consisting of corn, whole wheat, flax seed, and rice.

(3) In another variant the natural hot water bottle substitute further includes aromatherapy materials selected from the group consisting of chamomile, cinnamon, lemongrass, peppermint, rosemary, spearmint, white-willow, yarrow-flowers, yellow dock root and lavender.

(4) In yet another variant, the temperature retaining material are 65%-100% of materials secured between the front and back walls.

(5) In still another variant, the aromatherapy materials are 15% to 35% of materials secured between the front and back walls.

(6) In a further variant, the natural hot water bottle substitute further includes a fitted cover. The cover is formed in the shape of the natural hot water bottle substitute and has a front panel, a rear panel and an access covering panel. The front panel extends from a top end to a bottom end. The rear panel is substantially similar to the front panel and attached to the front panel at the top end, and along side edges of the front panel, leaving an opening along the bottom end. The access covering panel is attached to the bottom end of the front panel and side edges of the front and rear panels and extends upwardly for a first predetermined distance from the bottom end. The access covering panel covers the opening along the bottom end and secures the natural hot water bottle substitute within the fitted cover.

(7) In a final variant of the invention, the fitted cover is formed of material selected from the group consisting of microfiber material, fabric, plastic, and leather.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a partially cut-away perspective view of the preferred embodiment of the invention disposed within a washable cover;

FIG. 2 is a side elevation of the FIG. 1 embodiment in the washable cover;

FIG. 3 is a partially cut-away perspective view of the FIG. 1 embodiment illustrating the heat retaining/aromatherapy materials disposed within the container;

FIG. 4 is a partially cut-away perspective view of the FIG. 1 embodiment illustrating the placement of the fastenings and the control of the heat retaining/aromatherapy materials;

FIG. 5 partially cut-away perspective detailed view of the washable cover illustrating the covered opening.

### DETAILED DESCRIPTION OF THE PREFERRED EMBODIMENT

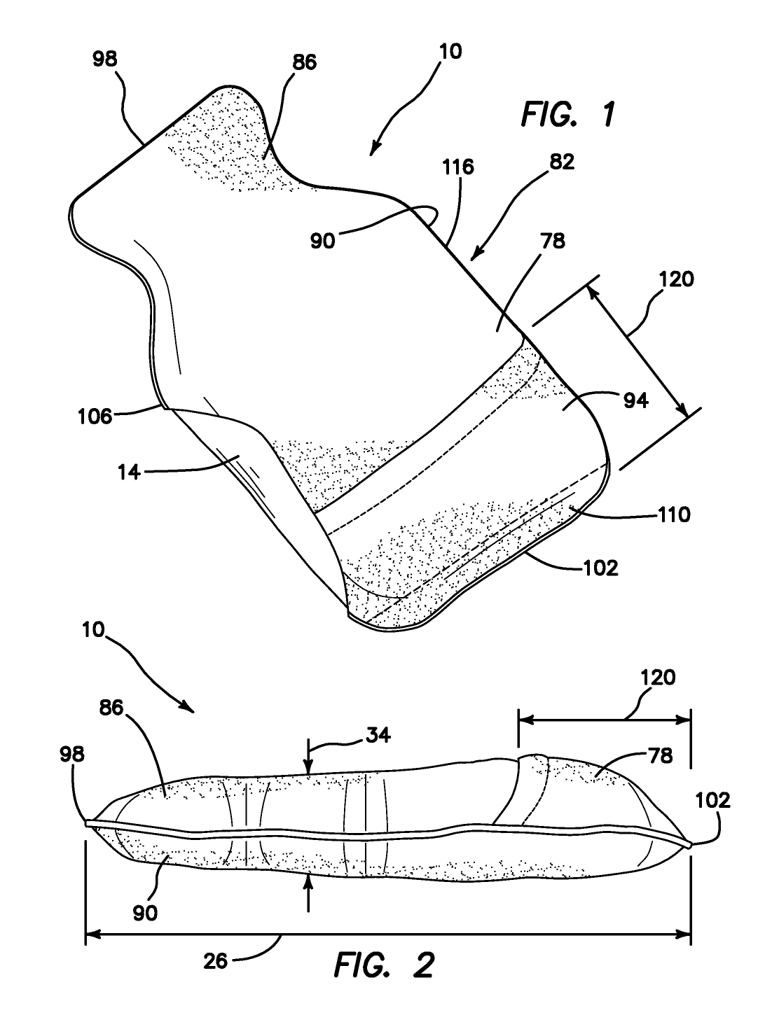

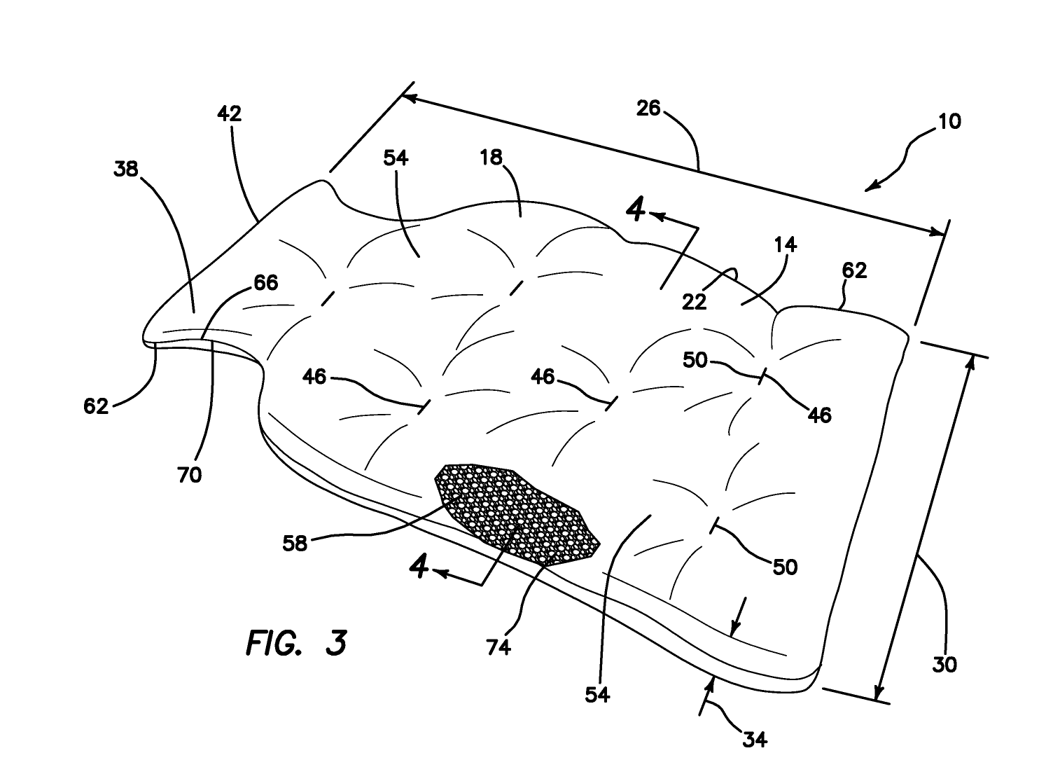

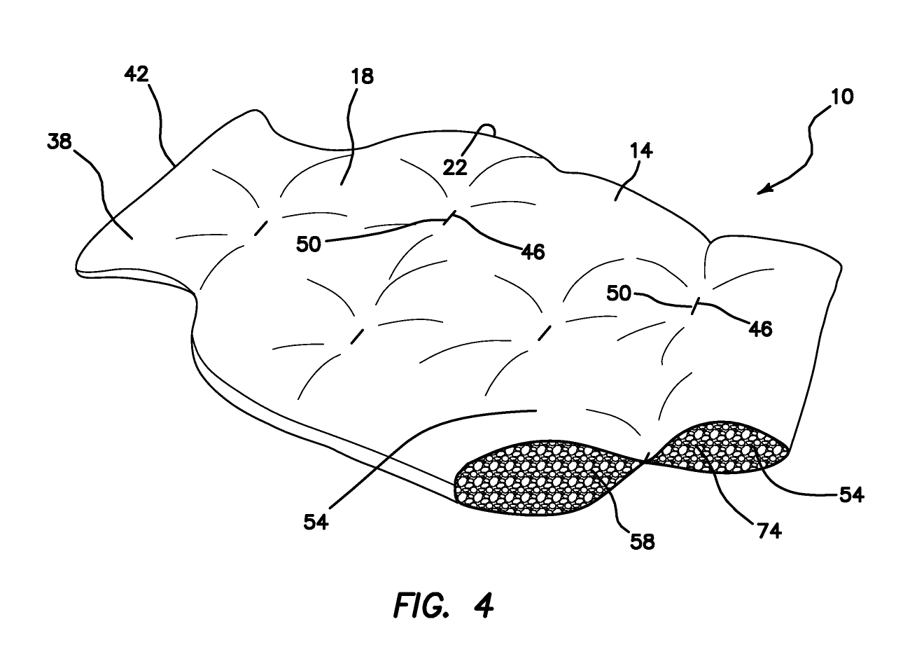

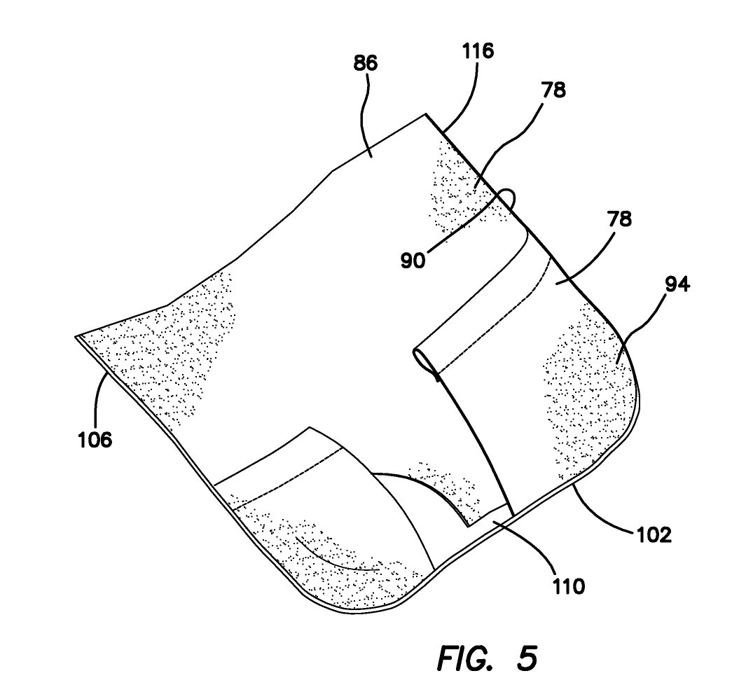

(1) FIGS. 1-5 illustrate a natural hot water bottle substitute 10 providing all of the desired features that can be constructed from the following components. As shown in FIGS. 3 and 4, a container 14 formed of flexible, porous material is provided. The container 14 has a front wall 18, a back wall 22, a first predetermined length 26, a first predetermined width 30, a first predetermined thickness 34 and a handle portion 38 disposed at an upper end 42 of the front 18 and back 22 walls. A plurality of fastenings 46 is provided. The fastenings 46 attach the front wall 18 to the back wall 22 at a series of point locations 50 while providing a continuous open space 54 around the fastenings 46. Granular, temperature retaining material 58 is provided. The material 58 is located within the continuous open space 54. Stitching 62 is provided. The stitching 62 fastens a surrounding edge 66 of the front wall 18 to a surrounding edge 70 of the back wall 22 and enclosing the temperature retaining material 58.

(2) In a variant of the invention, the temperature retaining material 58 is selected from the group consisting of corn, whole wheat, flax seed, and rice.

(3) In another variant the natural hot water bottle substitute 10 further includes aromatherapy materials 74 selected from the group consisting of chamomile, cinnamon, lemongrass, peppermint, rosemary, spearmint, white-willow, yarrow-flowers, yellow dock root and lavender.

(4) In yet another variant, the temperature retaining material 58 are 65%-100% of materials secured between the front 18 and back 22 walls.

(5) In still another variant, the aromatherapy materials 74 are 15% to 35% of materials secured between the front 18 and back 22 walls.

(6) In a further variant, as illustrated in FIGS. 1, 2 and 5, the natural hot water bottle substitute 10 further includes a fitted cover 78. The cover 78 is formed in the shape 82 of the natural hot water bottle substitute 10 and has a front panel 86, a rear panel 90 and an access covering panel 94. The front panel 86 extends from a top end 98 to a bottom end 102. The rear panel 90 is substantially similar to the front panel 86 and attached to the front panel 86 at the top end 98, and along side edges 106 of the front panel 86, leaving an opening 110 along the bottom end 102. The access covering panel 94 is attached to the bottom end 102 of the front panel 86 and side edges 106, 116 of the front 86 and rear 90 panels and extends upwardly for a first predetermined distance 120 from the bottom end 102. The access covering panel 94 covers the opening 110 along the bottom end 102 and secures the natural hot water bottle substitute 10 within the fitted cover 78.

(7) In a final variant of the invention, the fitted cover 78 is formed of material selected from the group consisting of microfiber material, fabric, plastic, and leather.

The natural hot water bottle substitute 10 has been described with reference to particular embodiments. Other modifications and enhancements can be made without departing from the spirit and scope of the claims that follow.

1. A natural hot water bottle substitute, comprising:
a container formed of flexible, porous material, said container having a front wall, a back wall, a first predetermined length, a first predetermined width, a first predetermined thickness and a handle portion disposed at an upper end of said front and back walls;
a plurality of fastenings, said fastenings attaching said front wall to said back wall at a series of point locations while providing a continuous open space around said fastenings;
granular, temperature retaining material, said material disposed within said continuous open space;
stitching, said stitching fastening a surrounding edge of said front wall to a surrounding edge of said back wall and enclosing said temperature retaining material.

2. The natural hot water bottle substitute, as described in claim 1, wherein said temperature retaining material is selected from the group consisting of:
corn, whole wheat, flax seed, and rice.

3. The natural hot water bottle substitute, as described in claim 1, further comprising aromatherapy materials selected from the group consisting of:
chamomile, cinnamon, lemongrass, peppermint, rosemary, spearmint, white-willow, yarrow-flowers, yellow dock root and lavender.

4. The natural hot water bottle substitute, as described in claim 1, wherein said temperature retaining material comprises 65%-100% of materials secured between said front and back walls.

5. The natural hot water bottle substitute, as described in claim 3, wherein said aromatherapy materials comprise 15% to 35% of materials secured between said front and back walls.

6. The natural hot water bottle substitute, as described in claim 1, further comprising:
a fitted cover, said cover being formed in the shape of said natural hot water bottle substitute and having a front panel, a rear panel and an access covering panel;
said front panel extending from a top end to a bottom end, said rear panel being substantially similar to said front panel and attached to said front panel at said top end, and along side edges of said front panel, leaving an opening along said bottom end;
said access covering panel being attached to said bottom end of said front panel and side edges of said front and rear panels and extending upwardly for a first predetermined distance from said bottom end; and
said access covering panel covering said opening along said bottom end and securing said natural hot water bottle substitute within said fitted cover.

7. The natural hot water bottle substitute, as described in claim 6, wherein said fitted cover is formed of material selected from the group consisting of:
microfiber material, fabric, plastic, and leather.
